SEQUENCE LISTING (10 sequences)

SEQ ID NO: 1 - DNA, 1118 bp, Enterobacteriaceae bacterium
  misc_feature (2)..(7): n is a, c, g, or t
  misc_feature (9)..(9): n is a, c, g, or t
  misc_feature (11)..(11): n is a, c, g, or t
  misc_feature (13)..(14): n is a, c, g, or t
  misc_feature (17)..(18): n is a, c, g, or t
  misc_feature (28)..(31): n is a, c, g, or t
  misc_feature (750)..(750): n is a, c, g, or t
tnnnnnnmncncnngcnnagagtctaannnnmcgtacttcttttgcaacccactcccatggtgtgacgggcggtgtgtacaaggcccgggaacgtattcaccgtagcattctgatctacgattactagcgattccgacttcacggagtcgagttgcagactccgatccggactacgacgcactttatgaggtccgcttgctctcgcgaggtcgcttctctttgtatgcgccattgtagcacgtgtgtagccctactcgtaagggccatgatgacttgacgtcatccccaccttcctccagtttatcactggcagtctcctttgagttcccggccgaaccgctggsaacaaaggataagggttgcgctcgttgcgggacttaacccaacatttcacaacacgagctgacgacmgccatgsagcacctgkctcagarttcccgaaggsaccaaagcatctctgctaagttctctggatgtcaagartargtaaggktcttcscgttgcatcsaawtaaaccacatgstccaccgcttgkgcgggcccccgtcmattcatttgagttttaaccttgcggccgtactccccaggsggtcgacttaacgcgttagctccggaagccactcctcaagggaacaacctccaagtcgacatcgtttacggsgtggactaccagggtawctaatcctgkttgctccccacgctttcscacctgarcgtcagtctttgtccagggggccgccttcscccnggtattcctccmgawctctacgcatttcacgctaccctggaattctaccccctctacagactctgcctgscagttcsaagsagtcccagktgagcccgggattcaatccgactgacgaccgccgcgkggcttacgccagtaattcgattacgctgcacctcctataccgcgctgcggcasgagtagccgtgctcttctgcggtacgtcatcgtgaactataattcacgcttctcccatgaagtacttacacccgaagcttctcwammgcgctggtgctcagctgcgccatggcatatcccmtgcgctccctagartyggacgggyycattccgkkggtggcwccyycgacactaggackccccagtga

SEQ ID NO: 2 - DNA, 1168 bp, Pantoea sp. strain SPC23
  misc_feature (2)..(3): n is a, c, g, or t
  misc_feature (5)..(8): n is a, c, g, or t
  misc_feature (11)..(17): n is a, c, g, or t
  misc_feature (21)..(23): n is a, c, g, or t
  misc_feature (26)..(31): n is a, c, g, or t
  misc_feature (34)..(37): n is a, c, g, or t
  misc_feature (39)..(39): n is a, c, g, or t
  misc_feature (1031)..(1031): n is a, c, g, or t
  misc_feature (1161)..(1163): n is a, c, g, or t
  misc_feature (1167)..(1167): n is a, c, g, or t
gnngnnnnctnnnnnnncccnnncgnnnnnnaannnntnstacttcttttgcaacccactcccatggtgtgacgggcggtgtgtacaaggcccgggaacgtattcaccgtggcattctgatccacgattactagcgattccgacttcacggagtcgagttgcagactccgatccggactacgacgcactttgtgaggtccgcttgctctcgcgaggtcgcttctctttgtatgcgccattgtagcacgtgtgtagccctactcgtaagggccatgatgacttgacgtcatccccaccttcctccggtttatcaccggcagtctcctttgagttcccgaccgaatcgctggcaacaaaggataagggttgcgctcgttgcgggacttaacccaacatttcacaacacgagctgacgacagccatgcagcacctgtctcagcgttcccgaaggcaccaaggcatctctgccaagttcgctggatgtcaagagtaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccactcctcaagggaacaacctccaagtcgacatcgtttacggcgtggactaccagggtatctaatcctgtttgctccccacgctttcgcacctgagcgtcagtcttcgtccagggggccgccttcgccaccggtattcctccagatctctacgcatttcaccgctacacctggaattctacccccctctacgagactcaagcctgccagtttcaaatgcagttcccaggttaagcccggggatttcacatctgacttaacagaccgcctgcgtgcgctttacgcccagtaattccgattaacgcttgcaccctccgtattaccgcggctgctggcacggagttagccggtgcttcttctgcgggtaacgtcaatcgacgcggttattaaccgcatcgccttcctncccgctgaaagtactttacaacccgaaggccttcttcatacacgcggcatggctgcatcaggcttgcgcccattgkgcaatattcccactgctgcctcccgtaggartytggaccgggtyycaattccmnnnkggng

SEQ ID NO: 3 - DNA, 1371 bp, Kluyvera sp. strain PS20
  misc_feature (1)..(8): n is a, c, g, or t
  misc_feature (12)..(12): n is a, c, g, or t
  misc_feature (1194)..(1194): n is a, c, g, or t
  misc_feature (1197)..(1197): n is a, c, g, or t
  misc_feature (1247)..(1247): n is a, c, g, or t
  misc_feature (1288)..(1288): n is a, c, g, or t
  misc_feature (1291)..(1291): n is a, c, g, or t
  misc_feature (1313)..(1314): n is a, c, g, or t
  misc_feature (1331)..(1331): n is a, c, g, or t
  misc_feature (1336)..(1337): n is a, c, g, or t
  misc_feature (1339)..(1340): n is a, c, g, or t
  misc_feature (1353)..(1354): n is a, c, g, or t
nnnnnnnnaagnggtagcgccctcccgaaggttaagctacctacttcttttgcaacccactcccatggtgtgacgggcggtgtgtacaaggcccgggaacgtattcaccgtagcattctgatctacgattactagcgattccgacttcacggagtcgagttgcagactccgatccggactacgacgcactttatgaggtccgcttgctctcgcgaggtcgcttctctttgtatgcgccattgtagcacgtgtgtagccctactcgtaagggccatgatgacttgacgtcatccccaccttcctccagtttatcactggcagtctcctttgagttcccggccgaaccgctggcaacaaaggataagggttgcgctcgttgcgggacttaacccaacatttcacaacacgagctgacgacagccatgcagcacctgtctcagagttcccgaaggcaccaaagcatctctgctaagttctctggatgtcaagagtaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccactcctcaagggaacaacctccaagtcgacatcgtttacggcgtggactaccagggtatctaatcctgtttgctccccacgctttcgcacctgagcgtcagtctttgtccagggggccgccttcgccaccggtattcctccagatctctacgcatttcaccgctacacctggaattctacccccctctacaagactctagcctgccagtttcgaatgcagttcccaggttgagcccggggatttcacatccgacttgacagaccgccggcgtgcgctttacgcccagtaattccgattaacgcttgcaccctccgtattaccgcggctgctggcacggagttagccggtgcttcttctgcgggtaacgtcaatcggtgaagctattaacttcaccgccttcctccccactgaaagtactttacaacccgaaggccttcttcatacacgcggcatggctgcatcaggcttgcgcccattgtgcatattcccactgctgctcccgtagaatctggaccgtgtctcatttccgtgtggctggtcatcctctcaaaccagctagggatcntcnccaggtaagcattacccacctacaagctatccatcgggccatccaaggtntgaggcccaaggcccccctttggcttggaactttgcggatnagnaccgtttccgaatttccccccnnggggatttccaaattcnaccgnncnngtcccaggaaacnnttgccccccatagtagc

SEQ ID NO: 4 - DNA, 1234 bp, Enterobacter ludwigii strain SNU502
  misc_feature (1)..(9): n is a, c, g, or t
  misc_feature (15)..(16): n is a, c, g, or t
  misc_feature (967)..(967): n is a, c, g, or t
  misc_feature (1140)..(1140): n is a, c, g, or t
  misc_feature (1177)..(1177): n is a, c, g, or t
  misc_feature (1208)..(1210): n is a, c, g, or t
  misc_feature (1217)..(1218): n is a, c, g, or t
  misc_feature (1222)..(1222): n is a, c, g, or t
  misc_feature (1228)..(1229): n is a, c, g, or t
  misc_feature (1232)..(1233): n is a, c, g, or t
nnnnnnnnncctacnntgcagtcgaacggtagcacagagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggtgttgtggttaataaccgcagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttnccagagatgctttggtgccttcgggaactctgagacaggtgctgcatggctgtcgtcagctcgtgttgtgaaatgtttgggttaagtcccgcaacgagcgcaacccttatcctttgttgccggcggtccggccgggaactcaaaggagactgccagtgataaactggaggaanggtggggatgacgtcaagtcatcagggcccttacaantagggctacacacttgcttcaatggcccaannnaaaaaannaaanccccgnngcnnc

SEQ ID NO: 5 - DNA, 1249 bp, Entrobacter ludwigi strain SNU502
  misc_feature (1)..(11): n is a, c, g, or t
  misc_feature (14)..(14): n is a, c, g, or t
  misc_feature (30)..(30): n is a, c, g, or t
  misc_feature (33)..(33): n is a, c, g, or t
  misc_feature (39)..(41): n is a, c, g, or t
  misc_feature (971)..(971): n is a, c, g, or t
  misc_feature (1190)..(1191): n is a, c, g, or t
  misc_feature (1221)..(1221): n is a, c, g, or t
  misc_feature (1228)..(1231): n is a, c, g, or t
  misc_feature (1242)..(1243): n is a, c, g, or t
  misc_feature (1247)..(1248): n is a, c, g, or t
nnnnnnnnnnnacncatgcagttcgtacgngtngtcacnnnagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggtgttgtggttaataaccgcagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttnccagagatgctttggtgccttcgggaactctgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaagtggggatgacgtcaagtcatcagggcccttaccagtagggctaccnncgtgctacaatggcgcaaacaaagaaaacnaattccnnnncagcggacctnnaaanng

SEQ ID NO: 6 - DNA, 1161 bp, Lelliottia amnigenastrain NCTC12124
  misc_feature (1)..(6): n is a, c, g, or t
  misc_feature (11)..(11): n is a, c, g, or t
nnnnnnaaagnggtaagcgccctccgaaggttaagctacctacttcttttgcaacccactcccatggtgtgacgggcggtgtgtacaaggcccgggaacgtattcaccgtagcattctgatctacgattactagcgattccgacttcacggagtcgagttgcagactccgatccggactacgacatactttgtgaggtccgcttgctctcgcgaggtcgcttctctttgtatatgccattgtagcacgtgtgtagccctactcgtaagggccatgatgacttgacgtcatccccaccttcctccagtttatcactggcagtctcctttgagttcccggccgaaccgctggcaacaaaggataagggttgcgctcgttgcgggacttaacccaacatttcacaacacgagctgacgacagccatgcagcacctgtctcagagttcccgaaggcaccaaagcatctctgctaagttctctggatgtcaagagtaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccactcctcaagggaacaacctccaagtcgacatcgtttacggcgtggactaccagggtatctaatcctgtttgctccccacgctttcgcacctgagcgtcagtctttgtccagggggccgccttcgccaccggtattcctccagatctctacgcatttcaccgctacacctggaattctacccccctctacaagactctagcctgccagtttcgaatgcagttcccaggttgagccccggggatttcacatccgacttgacagaccgcccggcgtgcgctttacgcccagtaattccgattaacgcttgcaccctccgtattaccgcggctgctggcacggagttagccggtgcttcttctgcgagtaacgtcaatcactgcggttattaaccacaatgcccttcctcctcgctgaaagtactttacaacccgaaaggccttcttcatacacggcggcatggctgcatcaggcttggcgcccattggtgcaatattccccactgctgccttcccgtaggaattctggaaccgtgt

SEQ ID NO: 7 - DNA, 1315 bp, Klebsiella sp. strain MWX2
  misc_feature (2)..(5): n is a, c, g, or t
  misc_feature (9)..(11): n is a, c, g, or t
  misc_feature (14)..(14): n is a, c, g, or t
  misc_feature (41)..(43): n is a, c, g, or t
  misc_feature (45)..(45): n is a, c, g, or t
  misc_feature (440)..(440): n is a, c, g, or t
  misc_feature (1195)..(1196): n is a, c, g, or t
  misc_feature (1208)..(1208): n is a, c, g, or t
  misc_feature (1231)..(1231): n is a, c, g, or t
  misc_feature (1251)..(1251): n is a, c, g, or t
  misc_feature (1270)..(1270): n is a, c, g, or t
  misc_feature (1278)..(1278): n is a, c, g, or t
  misc_feature (1281)..(1281): n is a, c, g, or t
  misc_feature (1300)..(1300): n is a, c, g, or t
gnnnnggcnnngcnacacatgcagtcgtaccagtgagtcannnanggagcttgctctcgggtgacgagtggcggacgggtgagtaatgtctgggaaactgcctgatggagggggataactactggaaacggtagctaataccgcataacgtcgcacgaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggtgatgaggttaataacctcntcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctggagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccagtgataactggaggaaggtggggatgacgtccagtcatcatggcccttacgagtagggctaccacnngctacatggcanatacaagaagaacgacctcgcgngagcagcggactcataagtngtcctaatccggatggatntgcactcnacncctgaagtcgaatcgctanaatcgggatcaaagc

SEQ ID NO: 8 - DNA, 1172 bp, Entrobacter ludwigi strain DM27
  misc_feature (1)..(9): n is a, c, g, or t
  misc_feature (12)..(14): n is a, c, g, or t
  misc_feature (17)..(18): n is a, c, g, or t
  misc_feature (23)..(23): n is a, c, g, or t
  misc_feature (26)..(29): n is a, c, g, or t
  misc_feature (1164)..(1166): n is a, c, g, or t
  misc_feature (1171)..(1172): n is a, c, g, or t
nnnnnnnnnccnnngcnnagatnaannnnmctacttcttttgcaacccactcccatggtgtgacgggcggtgtgtacaaggcccgggaacgtattcaccgtagcattctgatctacgattactagcgattccgacttcatggagtcgagttgcagactccaatccggactacgacgcactttatgaggtccgcttactctcgcaagttcgcttctctttgtatgcgccattgtagcacgtgtgtagccctactcgtaagggccatgatgacttgacgtcatccccaccttcctccagtttatcactggcagtctcctttgagttcccggccgaaccgctggcaacaaaggataggggttgcgctcgttgcgggacttaacccaacatttcacaacacgagctgacgacagccatgcagcacctgtctcagagttcccgaaggcaccaawscatctctgswaagttctctggatgtcaagagtaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccacgcctcaagggcacaacctccaagtcgacatcgtttacagcgtggactaccagggtatctaatcctgtttgctccccacgctttcgcacctgagcgtcagtcttcgtccagggggccgccttcgccaccggtattcctccagatctctacgcatttcaccgctacacctggaattctacccccctctacgagactctagcttgccagtttcgaatgcagttcccaggttaagcccggggatttcacatccgacttgacaaaccgcctgcgtgcgctttacgcccagtaattccgattaacgcttgcaccctccgtattaccgcggctgctggcacggagttagccggtgcttcttctgcgagtaacgtcaatcggcaaggttattaaccttaccgccttcctcctcgctgaaagtactttacaacccgaaggccttcttcatacacgcggcatggctgcatcaggcttgcgcccattgkgcaatattccccactgctgccycccgtaggartctggaccgkgtyycaattcccgggkggctgggnnncyytnn

SEQ ID NO: 9 - DNA, 1092 bp, Pectobacterium cypripedii strain ICMP 1591
  misc_feature (2)..(6): n is a, c, g, or t
  misc_feature (8)..(10): n is a, c, g, or t
  misc_feature (12)..(12): n is a, c, g, or t
  misc_feature (14)..(15): n is a, c, g, or t
  misc_feature (19)..(20): n is a, c, g, or t
  misc_feature (25)..(25): n is a, c, g, or t
  misc_feature (30)..(32): n is a, c, g, or t
  misc_feature (34)..(34): n is a, c, g, or t
  misc_feature (36)..(36): n is a, c, g, or t
tnnnnntnnncncnngccnnagaknctaannnwnmntacttcttttgcaacccactcccatggtgtgacgggcggtgtgtacaaggcccgggaacgtattcaccgtagcattctgatctacgattactagcgattccgacttcatggagtcgagttgcagactccaatccggactacgacgcactttatgaggtccgcttgctctcgcgaggtcgcttctctttgtatgcgccattgtagcacgtgtgtagccctggccgtaagggccatgatgacttgacgtcatccccaccttcctccggtttatcaccggcagtctcctttgagttcccgaccgtatcgctggcaacaaaggataagggttgcgctcgttgcgggacttaacccaacatttcacaacacgagctgacgacagccatgcagcacctgtctcacagttcccgaaggcacyaaagcatctctgctaarttctstggatgtcaaggccaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccacgcctcaagggcacaacctccaagtcgacatcgtttacggcgtggactaccagggtatctaatcctgtttgctccccacgctttcgcacctgagcgtcagtcttcgtccagggggccgccttcgccaccggtattcctccagatctctacgcatttcaccgctacacctggaattctacccccctctacgagactccagcctgccagtttcgaatgcagttcccaggttaagcccggggatttcacatccgacttgacagaccgcctgcgtgcgctttacgcccagtaattccgattaacgcttgcaccctccgtattaccgcggctgctggcacggagttagccggtgcttcttctgcgggtaacgtcaatgmatgaagstattaacttcamacccttcctccccgctgaaagtactttacaacccgaaggccttcttcatacacgcggcatggctgcatcaggctg

SEQ ID NO: 10 - DNA, 1354 bp, Stenotrophomonas sp. G4
  misc_feature (1)..(13): n is a, c, g, or t
  misc_feature (16)..(16): n is a, c, g, or t
  misc_feature (1197)..(1197): n is a, c, g, or t
  misc_feature (1221)..(1221): n is a, c, g, or t
  misc_feature (1251)..(1251): n is a, c, g, or t
  misc_feature (1253)..(1253): n is a, c, g, or t
  misc_feature (1278)..(1279): n is a, c, g, or t
  misc_feature (1311)..(1311): n is a, c, g, or t
  misc_feature (1322)..(1322): n is a, c, g, or t
  misc_feature (1346)..(1347): n is a, c, g, or t
nnnnnnnnnnnnncgnggcagcgccctcccgaaggttaagctacctgcttctggtgcaacaaactcccatggtgtgacgggcggtgtgtacaaggcccgggaacgtattcaccgcagcaatgctgatctgcgattactagcgattccgacttcatggagtcgagttgcagactccaatccggactgagatggggtttctgggattggcttaccgtcgccggcttgcagccctctgtccccaccattgtagtacgtgtgtagccctggccgtaagggccatgatgacttgacgtcatccccaccttcctccggtttgtcaccggcggtctccttagagttcccaccattacgtgctggcaactaaggacaagggttgcgctcgttgcgggacttaacccaacatctcacgacacgagctgacgacagccatgcagcacctgtgttcgagttcccgaaggcacccatccatctctggaaagttctcgacatgtcaaggccaggtaaggttcttcgcgttgcatcgaattaaaccacatactccaccgcttgtgcgggcccccgtcaattcctttgagtttcagtcttgcgaccgtactccccaggcggcgaacttaacgcgttagcttcgatactgcgtgccaaattgcacccaacatccagttcgcatcgtttagggcgtggactaccagggtatctaatcctgtttgctccccacgctttcgtgcctcagtgtcagtgttggtccaggtagctgccttcgccatggatgttcctcccgatctctacgcatttcactgctacaccgggaattccgctaccctctaccacactctagttgtccagtttccactgcagttcccaggttgagcccagggctttcacaacagacttaaacaaccacctacgcacgctttacgcccagtaattccgagtaacgcttgcacccttcgtattaccgcggctgctggcacgaagttagccggtgcttattctttgggtaccgtcatcccaaccaggtattagccggctggatttctttcccaacaaaagggctttacaacccgaaggccttcttcacccacgcggtatggctggatcaggcttgcgcccattgtccatattccccactgctgcctccgtaggaatctggacgtgtctcatttccgtgtggctgatcatctctcaaacagctacggntcgtcgcctggtgggctttacccnccactagctaatcgaaatcggtcattcatngncaaggccgaagtccctgctttcccnnaggtcgaagcggattagcgaatttcctacttnccccccaaaantaattcaaatatttctacctttgnnaatggca

(19) United States
(12) Patent Application Publication
(10) Pub. No.: US 2021/0261476 A1
(43) Pub. Date: Aug. 26, 2021

(54) MULTI-FUNCTIONAL BIOFERTILIZER

(71) Applicants: Ali Hendiyani, Qom (IR); Ebrahim Eshaghi, Qom (IR); Sara Mousaei, Qom (IR); Rahim Nosrati, Gilan (IR)

(72) Inventors: Ali Hendiyani, Qom (IR); Ebrahim Eshaghi, Qom (IR); Sara Mousaei, Qom (IR); Rahim Nosrati, Gilan (IR)

(73) Assignee: Aria Nahan Zist Exir, Qom (IR)

(21) Appl. No.: 17/175,702

(22) Filed: Feb. 15, 2021

Related U.S. Application Data
(60) Provisional application No. 62/981,051, filed on Feb. 25, 2020.

(51) Int. Cl. C05F 11/08; C12N 1/20

(52) U.S. Cl. CPC C05F 11/08; C12R 2001/01; C12N 1/205

(57) ABSTRACT

An exemplary biofertilizer may include a first component that may include an isolated bacterium blended with a first carrier. An exemplary isolated bacterium may include a nucleic acid sequence identical to at least one of SEQ ID NO. 1 and SEQ ID NO. 3. An exemplary biofertilizer may further include a second component including a pair of isolated bacteria blended with a second carrier. An exemplary pair of isolated bacteria may include nucleic acid sequences identical to nucleic acid sequences selected from the group consisting of SEQ ID NO. 1, SEQ ID NO. 3, and SEQ ID NO. 5.

### CROSS-REFERENCE TO RELATED APPLICATION

This application claims the benefit of priority from pending U.S. Provisional Patent Application Ser. No. 62/981,051, filed on Feb. 25, 2020, and entitled “MULTI-FUNCTIONAL BIOFERTILIZER FOR NITROGEN (N), PHOSPHORUS (P), POTASSIUM (K) AND PLANT ROOT STIMULATION,” which is incorporated herein by reference in its entirety.

### TECHNICAL FIELD

The present disclosure relates to biofertilizers, and more particularly relates to multi-functional biofertilizers.

### BACKGROUND

Fertilizers are natural or synthetic materials that may be applied to soil or plant tissues. Fertilizers may contain chemical elements, such as nitrogen, phosphorus, potassium, and plant growth hormones that are necessary for improving growth and productiveness of plants.

Fertilizers include inorganic fertilizers, organic fertilizers, and biofertilizers. Organic fertilizers may be produced as an end product or a byproduct of naturally occurring processes in nature. Inorganic fertilizers are synthetic materials which may only contain a few nutrients such as nitrogen, phosphorus, and potassium. Inorganic fertilizers may deteriorate the fertility of the soil in a long-term usage due to the increase of soil acidity. Nutrients in inorganic fertilizers may penetrate from soil to flowing water and endanger aquatic life. These nutrients may also enter drinking water and jeopardize human health. Furthermore, excess inorganic ingredients in nature may cause severe diseases such as blue baby syndrome, cancer, and chronic diseases.

To address these problems and still supply the demand of the growing population, biofertilizers may be utilized. Biofertilizers may contain micro-organisms and plant remains. Biofertilizers are cost-effective and eco-friendly fertilizers that may supply necessary nutrients for plants and enhance the productivity of crops. Biofertilizers may also produce antibiotics, which may protect plants against pathogens. One drawback of available biofertilizers may be that most biofertilizers may only supply a single nutrient for plants, and as a result for supplying more than one nutrient, more than one type of biofertilizer must be used.

There is, therefore, a need for new compositions of biofertilizers that may supply necessary nutrients such as nitrogen, phosphorus, potassium, and plant growth hormones, simultaneously.

### SUMMARY

This summary is intended to provide an overview of the subject matter of the present disclosure and is not intended to identify essential elements or key elements of the subject matter, nor is it intended to be used to determine the scope of the claimed implementations. The proper scope of the present disclosure may be ascertained from the claims set forth below in view of the detailed description and the drawings.

According to one or more exemplary embodiments, the present disclosure is directed to an exemplary biofertilizer and a method for producing an exemplary biofertilizer. An exemplary biofertilizer may include a first component that may include an isolated bacterium blended with a first carrier. An exemplary isolated bacterium may include a nucleic acid sequence identical to at least one of SEQ ID NO. 1, SEQ ID NO. 2, SEQ ID NO. 3, SEQ ID NO. 8, and SEQ ID NO. 9. An exemplary biofertilizer may further include a second component including a pair of isolated bacteria blended with a second carrier. An exemplary pair of isolated bacteria may include nucleic acid sequences identical to nucleic acid sequences selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, and SEQ ID NO. 10.

In an exemplary embodiment, an exemplary biofertilizer may include an exemplary first carrier. An exemplary first carrier may include 2-3 wt. % of a corn steep liquor, based on an exemplary total weight of an exemplary first component, 0.5-1 wt. % of sugar beet molasses, based on an exemplary total weight of an exemplary first component, 1-10 wt. % of glycerin, based on an exemplary total weight of an exemplary first component, and water.

In an exemplary embodiment, an exemplary first component may include 0.5 wt. % to 10 wt. % of an exemplary isolated bacterium based on an exemplary total weight of an exemplary first component.

In an exemplary embodiment, an exemplary second carrier may include 2-3 wt. % of a corn steep liquor, based on an exemplary total weight of an exemplary second component, 0.5-1 wt. % of sugar beet molasses, based on an exemplary total weight of an exemplary second component, 1-10 wt. % of glycerin, based on an exemplary total weight of an exemplary second component, and water.

In an exemplary embodiment, an exemplary second component may include 0.5 wt. % to 10 wt. % of an exemplary pair of isolated bacteria based on an exemplary total weight of an exemplary second component.

In an exemplary embodiment, an exemplary first carrier may include 0.5-1.5 wt. % of pearlite, based on an exemplary total weight of an exemplary first component and 0.5-1.5 wt. % of bentonite, based on an exemplary total weight of an exemplary first component. An exemplary first carrier may also include a filler and water. An exemplary filler may include one of a first filler and a second filler. In an exemplary embodiment, an exemplary first filler may include 0.5-1 wt. % of soybean meal and 50 wt. % of bagasse, based on an exemplary total weight of an exemplary first component. An exemplary second filler may include 50 wt. % of soybean meal and 0.5-1 wt. % of bagasse, based on an exemplary total weight of an exemplary first component.

In an exemplary embodiment, an exemplary first component may include 1 wt. % to 10 wt. % of an exemplary isolated bacterium based on an exemplary total weight of an exemplary first component.

In an exemplary embodiment, an exemplary second carrier may include 0.5-1.5 wt. % of pearlite, based on an exemplary total weight of an exemplary second component, 0.5-1.5 wt. % of bentonite, based on an exemplary total weight of an exemplary second component, water, and a filler. An exemplary filler may include at least one of a first filler and a second filler. In an exemplary embodiment, an exemplary first filler may include 0.5-1 wt. % of soybean meal and 50 wt. % of bagasse, based on an exemplary total weight of an exemplary second component. An exemplary second filler may include 50 wt. % of soybean meal and 0.5-1 wt. % of bagasse, based on an exemplary total weight of an exemplary second component.

In an exemplary embodiment, an exemplary second component may include 1 wt. % to 10 wt. % of an exemplary pair of isolated bacteria based on an exemplary total weight of an exemplary second component.

In an exemplary embodiment, an exemplary isolated bacterium may include at least one of Enterobacteriaceae bacterium, pantoea sp. strain SPC23, kluyvera sp. strain PS20, Enterobacter ludwigii strain DM27, and pectobacterium cypripedii strain ICMP 1591.

In an exemplary embodiment, an exemplary pair of bacteria may be selected from a group consisting of Enterobacteriaceae bacterium, pantoea sp. strain SPC23, kluyvera sp. strain PS20, Enterobacter ludwigi strain SNU502, Lelliottia amnigena strain NCTC12124, klebsiella sp. strain MWX2, Enterobacter ludwigii strain DM27, pectobacterium cypripedii strain ICMP 1591, and stenotrophomonas sp. G4.

In an exemplary embodiment, an exemplary method for producing an exemplary biofertilizer may include forming a first component by forming a first mixture by blending an isolated bacterium with a first carrier, heating the first mixture at a temperature between 28° C. and 30° C., forming a second component by forming a second mixture by blending a pair of isolated bacteria with a second carrier, heating the second mixture at a temperature between 28° C. and 30° C., and mixing the first component and the second component.

In an exemplary embodiment, an exemplary first mixture may be obtained by blending a first carrier with an exemplary isolated bacterium including a nucleic acid sequence identical to at least one of SEQ ID NO. 1, SEQ ID NO. 2, SEQ ID NO. 3, SEQ ID NO. 8, and SEQ ID NO. 9. In an exemplary embodiment, an exemplary second mixture may be obtained by blending a second carrier with a pair of isolated bacteria including nucleic acid sequences identical to nucleic acid sequences selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, and SEQ ID NO. 10.

In an exemplary embodiment, forming an exemplary first mixture may include blending an exemplary isolated bacterium with an exemplary first carrier with a concentration of an exemplary isolated bacterium between 1 wt. % and 10 wt. % of an exemplary total weight of an exemplary first component. In an exemplary embodiment, an exemplary first carrier may include 2-3 wt. % of a corn steep liquor, based on an exemplary total weight of an exemplary first component, 0.5-1 wt. % of sugar beet molasses, based on an exemplary total weight of an exemplary first component, 1-10 wt. % of glycerin, based on an exemplary total weight of an exemplary first component, and water.

In an exemplary embodiment, an exemplary second mixture may include blending an exemplary pair of isolated bacteria with an exemplary second carrier with a concentration of an exemplary pair of isolated bacteria between 1 wt. % and 10 wt. % of an exemplary total weight of an exemplary second component. In an exemplary embodiment, an exemplary second carrier may include 2-3 wt. % of a corn steep liquor, based on an exemplary total weight of an exemplary second component, 0.5-1 wt. % of sugar beet molasses, based on an exemplary total weight of an exemplary second component, 1-10 wt. % of glycerin, based on an exemplary total weight of an exemplary second component, and water.

In an exemplary embodiment, an exemplary method of producing an exemplary biofertilizer may further include adjusting pH of an exemplary first component and an exemplary second component at a pH between 7 and 9.

In an exemplary embodiment, forming an exemplary first mixture may include blending an exemplary isolated bacterium with an exemplary first carrier with a concentration of an exemplary isolated bacterium between 0.5 wt. % and 10 wt. % of an exemplary total weight of the first component.

In an exemplary embodiment, an exemplary first carrier may include 0.5-1.5 wt. % of pearlite, based on an exemplary total weight of an exemplary first component, 0.5-1.5 wt. % of bentonite, based on an exemplary total weight of an exemplary first component, water, and a filler. An exemplary filler may include a first filler and a second filler. In an exemplary embodiment, an exemplary first filler may include 0.5-1 wt. % of soybean meal and 50 wt. % of bagasse, based on an exemplary total weight of an exemplary first component. An exemplary second filler may include 50 wt. % of soybean meal and 0.5-1 wt. % of bagasse, based on the total weight of the first component.

In an exemplary embodiment, forming an exemplary second mixture may include blending an exemplary pair of isolated bacteria with an exemplary second carrier with a concentration of an exemplary pair of isolated bacteria between 0.5 wt. % and 10 wt. % of an exemplary total weight of an exemplary second component. In an exemplary embodiment, an exemplary second carrier may include 0.5-1.5 wt. % of pearlite, based on an exemplary total weight of an exemplary second component, 0.5-1.5 wt. % of bentonite, based on an exemplary total weight of an exemplary second component, water, and a filler. An exemplary filler may include at least one of a first filler and a second filler. In an exemplary embodiment, an exemplary first filler may include 0.5-1 wt. % of soybean meal and 50 wt. % of bagasse, based on an exemplary total weight of an exemplary second component. In an exemplary embodiment, an exemplary second filler may include 50 wt. % of soybean meal and 0.5-1 wt. % of bagasse, based on an exemplary total weight of an exemplary second component.

In an exemplary embodiment, an exemplary method of producing an exemplary biofertilizer may further include mixing an exemplary first component and an exemplary second component with a weight ratio of an exemplary first component to an exemplary second component between 0.5 and 2 (first component/second component).

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which a presently preferred embodiment of the present disclosure will now be illustrated by way of example. It is expressly understood, however, that the drawings are for illustration and description only and are not intended as a definition of the limits of the present disclosure. Embodiments of the present disclosure will now be described by way of example in association with the accompanying drawings in which:

FIG. 1 illustrates a flowchart of a method for producing a multi-functional biofertilizer, consistent with one or more exemplary embodiments of the present disclosure.

### DETAILED DESCRIPTION

The novel features which are believed to be characteristic of the present disclosure, as to its structure, organization, use and method of operation, together with further objectives and advantages thereof, will be better understood from the following discussion.

According to one or more exemplary embodiments, the present disclosure is directed to a new class of fertilizers that may supply various necessary nutrients for plants. In other words, the present disclosure is directed to exemplary embodiments of a multi-functional biofertilizer, in which different bacteria may be utilized as nutrient suppliers. Exemplary necessary nutrients of plants may include nitrogen, phosphorus, potassium, and plant growth hormones such as auxin. The present disclosure is further directed to exemplary embodiments of a multi-functional biofertilizer mixture that may include two mixtures of bacteria.

According to one or more exemplary embodiments, the present disclosure is further directed to exemplary embodiments of a method for producing biofertilizers. An exemplary method for producing a biofertilizer may include a step of cultivating bacteria. In an exemplary embodiment, exemplary bacteria may be extracted from rhizosphere and then the extracted bacteria may be cultivated in a culture medium placed inside an incubator for 24-48 hours. In an exemplary embodiment, pH, humidity, and temperature may be optimized and fixed in an exemplary incubator and may be between 7-9, 30%-40%, and 28° C.-30° C., respectively.

In an exemplary embodiment, an exemplary culture medium may include brain heart infusion broth (BHI Broth) or lysogeny broth (LB). Exemplary cultivated bacteria may then be transferred to a bioreactor to reach a number of viable bacteria equal to approximately 10⁶-10⁷ CFU. In an exemplary embodiment, an exemplary bioreactor may refer to a fermenter. In an exemplary embodiment, pH, humidity, and temperature may be optimized and fixed in an exemplary fermenter between 8-9, 30%-40%, and 28° C.-30° C., respectively. Exemplary cultivated bacteria may be stored, for example, at −80° C. and also by adding 25-30 wt. % glycerol into an exemplary cultivated media.

An exemplary biofertilizer may include a first component and a second component. An exemplary first component may include an exemplary isolated bacterium that may be blended with a first carrier. In an exemplary embodiment, an exemplary isolated bacterium may have a nucleotide sequence identical to at least one of SEQ ID NO. 1, SEQ ID NO. 2, SEQ ID NO. 3, SEQ ID NO. 8, and SEQ ID NO. 9. An exemplary first carrier may be a wet solid carrier that may include 0.5-1.5 wt. % of pearlite, 0.5-1.5 wt. % of bentonite, 50.5-51 wt. % of a filler, and water. In an exemplary embodiment, an exemplary filler may include one of a first filler including 0.5-1 wt. % of soybean meal and 50 wt. % of bagasse and a second filler including 50 wt. % of soybean meal and 0.5-1 wt. % of bagasse. In an exemplary embodiment, an exemplary first carrier may be a liquid carrier that may include 2-3 wt. % of a corn steep liquor, 0.5-1 wt. % of sugar beet molasses, 1-10 wt. % of glycerin, and water. In an exemplary embodiment, all weight percentages for the first carrier constitutes are based on the total weight of the first component. In an exemplary embodiment, a first component may include 1-10 wt. % of an exemplary isolated bacterium, based on the total weight of the first component, blended with an exemplary wet solid carrier. In an exemplary embodiment, a first component may include 0.5-10 wt. % of an exemplary isolated bacterium, based on the total weight of the first component, blended with an exemplary liquid carrier.

In an exemplary embodiment, an exemplary second component may include an exemplary isolated pair of bacteria and a second carrier. In an exemplary embodiment, an exemplary isolated pair of bacteria may include a pair of bacteria with nucleotide sequences identical to a respective pair of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, and SEQ ID NO. 10. An exemplary second carrier may be a wet solid carrier that may include 0.5-1.5 wt. % of pearlite, 0.5-1.5 wt. % of bentonite, 50.5-51 wt. % of a filler, and water. In an exemplary embodiment, an exemplary filler may include one of a first filler including 0.5-1 wt. % of soybean meal and 50 wt. % of bagasse and a second filler including 50 wt. % of soybean meal and 0.5-1 wt. % of bagasse. In an exemplary embodiment, an exemplary second carrier may be a liquid carrier that may include 2-3 wt. % of a corn steep liquor, 0.5-1 wt. % of sugar beet molasses, 1-10 wt. % of glycerin, and water. In an exemplary embodiment, all weight percentages of the second carrier constitutes are based on the total weight of the second component.

In an exemplary embodiment, a second component may include 1-10 wt. % of an exemplary pair of bacteria blended with an exemplary wet solid carrier. In an exemplary embodiment, a second component may include 0.5-10 wt. % of an exemplary pair of bacteria blended with an exemplary liquid carrier. As used herein, the weight percentage of bacteria within the second component are based on the total weight of the second component.

In an exemplary embodiment, an exemplary multifunctional biofertilizer may include a mixture of an exemplary first component and an exemplary second component with a weight ratio between 0.5 and 2 (first component to second component). In an exemplary embodiment, an exemplary multifunctional biofertilizer package may include two separate containers, one containing an exemplary first component and the other containing an exemplary second component. In an exemplary embodiment, the contents of the two separate containers may be mixed together right before being applied to the soil or plant tissues.

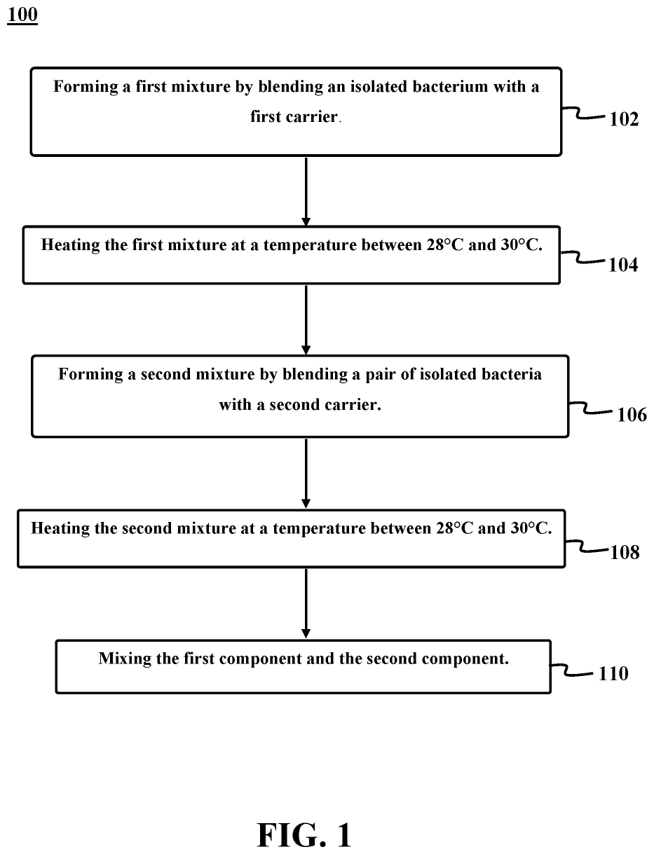

FIG. 1 illustrates a flowchart of a method 100 for producing a multi-functional biofertilizer, consistent with one or more exemplary embodiments of the present disclosure. In an exemplary embodiment, method 100 may include a step 102 of forming a first mixture by blending an isolated bacterium with a first carrier, a step 104 of heating the first mixture at a temperature between 28° C. and 30° C., a step 106 of forming a second mixture by blending a pair of isolated bacteria with a second carrier, a step 108 of heating the second mixture at a temperature between 28° C. and 30° C., and a step 110 of mixing the first component and the second component.

In an exemplary embodiment, step 102 of forming the first mixture may include blending an exemplary isolated bacterium with an exemplary first carrier, where an exemplary isolated bacterium may include a nucleic acid sequence identical to at least one of SEQ ID NO. 1, SEQ ID NO. 2, SEQ ID NO. 3, SEQ ID NO. 8, and SEQ ID NO. 9. Such exemplary isolated bacterium may be capable of fixing nitrogen, dissolving phosphorus and potassium, and producing auxin. In an exemplary embodiment, an exemplary isolated bacterium may include Enterobacteriaceae bacterium, pantoea sp. strain SPC23, kluyvera sp. strain PS20, Enterobacter ludwigii strain DM27, and pectobacterium cypripedii strain ICMP 1591. In an exemplary embodiment, blending an exemplary isolated bacterium with an exemplary first carrier may include pouring the exemplary isolated bacterium onto the exemplary carrier in a mechanical mixer, such as a blender and mixing the exemplary isolated bacterium and the exemplary carrier utilizing the blender.

In an exemplary embodiment, step 102 of forming the first mixture may include blending an isolated bacterium with a first carrier, where an exemplary first carrier may either be a liquid carrier or a wet solid carrier. An exemplary liquid carrier may include 2-3 wt. % of a corn steep liquor, 0.5-1 wt. % of sugar beet molasses, 1-10 wt. % of glycerin based on the total weight of the first mixture, and water. An exemplary wet solid carrier may include 0.5-1.5 wt. % of pearlite, 0.5-1.5 wt. % of bentonite, 50.5-51 wt. % of a filler, and water. An exemplary filler may include either 0.5-1 wt. % of soybean meal and 50 wt. % of bagasse or 50 wt. % of soybean meal and 0.5-1 wt. % of bagasse. In an exemplary embodiment, an exemplary first carrier, either a liquid or a wet solid carrier, may be formed by mixing all the above-mentioned constituents in a mechanical mixer. In an exemplary embodiment, step 102 of forming the first mixture may include blending a predetermined amount of isolated bacterium with a liquid carrier such that concentration of the isolated bacterium may be between 1 wt. % and 10 wt. % based on the total weight of the first mixture. In an exemplary embodiment, step 102 of forming the first mixture may include blending a predetermined amount of isolated bacterium with a wet solid carrier such that concentration of the isolated bacterium may be between 0.5 wt. % and 10 wt. % based on the total weight of the first mixture.

In an exemplary embodiment, step 104 of heating the first mixture at a temperature between 28° C. and 30° C. may include heating the first mixture during step 102 of forming the first mixture. In other words, step 104 of heating the first mixture may include heating the first mixture of an exemplary isolated bacterium and an exemplary carrier in a blender at a temperature between 28° C. and 30° C. In an exemplary embodiment, the pH of the first mixture may be fixed between 8 and 9 by adding KOH, NaOH, and HCl.

In an exemplary embodiment, step 106 of forming a second mixture by blending a pair of isolated bacteria with a second carrier, where an exemplary pair of bacteria may include nucleotide sequences identical to a respective pair of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, and SEQ ID NO. 10. Such exemplary pair of bacteria may be capable of fixing nitrogen, dissolving phosphorus and potassium, and producing auxin. In an exemplary embodiment, an exemplary pair of bacteria may include Enterobacteriaceae bacterium, pantoea sp. strain SPC23, kluyvera sp. strain PS20, Enterobacter ludwigi strain SNU502, lelliottia amnigena strain NCTC12124, klebsiella sp. strain MWX2, Enterobacter ludwigii strain DM27, pectobacterium cypripedii strain ICMP 1591, and stenotrophomonas sp. G4. In an exemplary embodiment, blending an exemplary pair of bacteria with an exemplary second carrier may include pouring the exemplary pair of bacteria onto the exemplary carrier in a mechanical mixer, such as a blender and mixing the exemplary pair of bacteria and the exemplary carrier utilizing the blender.

In an exemplary embodiment, step 106 of forming the second mixture may include blending a pair bacteria with a second carrier, where an exemplary second carrier may either be a liquid carrier or a wet solid carrier. An exemplary liquid carrier may include 2-3 wt. % of a corn steep liquor, 0.5-1 wt. % of sugar beet molasses, 1-10 wt. % of glycerin based on the total weight of the second mixture, and water. An exemplary wet solid carrier may include 0.5-1.5 wt. % of pearlite, 0.5-1.5 wt. % of bentonite, 50.5-51 wt. % of a filler, and water. An exemplary filler may include either 0.5-1 wt. % of soybean meal and 50 wt. % of bagasse or 50 wt. % of soybean meal and 0.5-1 wt. % of bagasse. In an exemplary embodiment, an exemplary second carrier, either a liquid or a wet solid carrier, may be formed by mixing all the above-mentioned constituents in a mechanical mixer. In an exemplary embodiment, step 106 of forming the second mixture may include blending a predetermined amount of isolated pair of bacteria with a liquid carrier such that concentration of isolated pair of bacteria may be between 1 wt. % and 10 wt. % based on the total weight of the second mixture. In an exemplary embodiment, step 106 of forming the second mixture may include blending a predetermined amount of the isolated pair of bacteria with a wet solid carrier such that concentration of the isolated pair of bacteria may be between 0.5 wt. % and 10 wt. % based on the total weight of the second mixture.

In an exemplary embodiment, step 108 of heating the second mixture at a temperature between 28° C. and 30° C. may include heating the second mixture during step 106 of forming the second mixture. In other words, step 108 of heating the second mixture may include heating the second mixture of an exemplary pair of bacteria and an exemplary carrier in a blender at a temperature between 28° C. and 30° C. In an exemplary embodiment, the pH of the second mixture may be fixed between 8 and 9 by adding KOH, NaOH, and HCl.

In an exemplary embodiment, step 110 of mixing the first component and the second component may include mixing an exemplary first component and an exemplary second component in a mixer. An exemplary mixer may include a container and rotating wings inside an exemplary container. An exemplary first component and an exemplary second component may be mixed in an exemplary mixer with a rotational speed of exemplary rotating wins between 80 rpm and 100 rpm for 3 minutes and 5 minutes, right before applying an exemplary biofertilizer to the soil or plant tissues. In an exemplary embodiment, an exemplary first component and an exemplary second component may be mixed in a weight ratio of between 0.5 and 2 (first component/second component).

### Example 1: Isolating Bacteria (Sample A)

Samples of agricultural soils were collected from the rhizosphere of various regions. Rhizosphere is a layer of soil at a depth of 5-20 cm from the ground surface. 1 g of each agricultural soil sample was added to 9 mL of a physiological serum. The suspension of agricultural soil sample and physiological serum was kept still for 30 minutes until the solid phase was settled down. 10 samples of different bacteria were collected and isolated for further analysis. Exemplary samples were named A₁ to A₁₀ and the isolated bacteria are shown in Table 1. Exemplary isolated bacteria were also analyzed for their biochemical properties and all the data are gathered in Table 2.

TABLE 1

| Sample | Accession number | Identified species | Similarity (%) |
|---|---|---|---|
| A₁ | Enterobacteriaceae bacterium | AB703079.1 | 100 |
| A₂ | Pantoea sp. strain SPC23 | MK053631.1 | 100 |
| A₃ | Kluyvera sp. strain PS20 | MH883998.1 | 99 |
| A₄ | Enterobacter ludwigii strain SNU502 | MK424123.1 | 100 |
| A₅ | Enterobacter ludwigii strain SNU502 | MK424123.1 | 100 |
| A₆ | Lelliottia amnigena strain NCTC12124 | LR134135.1 | 100 |
| A₇ | Klebsiella sp. strain MWX2 | MH105765.1 | 99 |
| A₈ | Enterobacter ludwigii strain DM27 | MK370572.1 | 99 |
| A₉ | Pectobacterium cypripedii strain ICMP | EF122434.1 | 99 |
| | 1591 | | |
| A₁₀ | Stenotrophomonas sp. G4 | CP031741.1 | 99 |

TABLE 2

| | Gram | | | Simmon | | | | | |
|---|---|---|---|---|---|---|---|---|---|
| Sample | staining | Catalase | Oxidase | citrate | Urea | H₂S | Lactose | Glucose | Mannitol |
| A₁ | − | + | − | − | − | − | + | + | + |
| A₂ | − | + | − | + | − | − | + | + | + |
| A₃ | − | + | − | + | − | − | + | + | + |
| A₄ | − | + | − | + | − | − | + | + | + |
| A₅ | − | + | − | + | − | − | + | + | + |
| A₆ | − | + | − | − | − | − | + | + | + |
| A₇ | − | + | − | + | − | − | + | + | + |
| A₈ | − | + | − | − | − | − | + | + | + |
| A₉ | − | + | − | + | − | − | + | + | + |
| A₁₀ | − | − | +/− | + | − | − | + | + | + |

### Example 2: Isolating Nitrogen Fixing Bacteria and Analyzing Nitrogen Fixing Ability of Bacteria

1 mL of sample A was added to 9 mL of a wet solid culture medium. The wet solid culture medium may include 20 g glucose and mannitol, 0.8 g K₂HPO₄, 0.2 g KH₂PO₄, 0.5 g MgSO₄.7H₂O, 0.05-0.1 g FeSO₄.6H₂O, 0.05 g CaCl₂.2H₂O and 20 g CaCO₃, 0.05 g NaMoO₄.2H₂O, and 5 g agar. Glucose and mannitol were added to 50 mL water and K₂HPO₄, KH₂PO₄, MgSO₄.7H₂O, FeSO₄.6H₂O, CaCl₂.2H₂O and CaCO₃, NaMoO₄.2H₂O, and agar were added to 950 mL water. The two mixtures were heated at 121° C. for 15 minutes to be sterilized. After sterilization process, two mixtures were mixed together, and the pH of the sample was kept constant between 7.2 and 7.3 utilizing KOH and HCl. Samples were heated at 28° C. and rotated at 100 rpm for 72-96 hours in an incubator. After passing the determined time, 100 μL of the sample was transferred to an N-free culture medium on a plate. The N-free culture medium may refer to a medium with no nitrogen. Then, the plates were heated for 72 to 48 hours at 28° C. Microorganisms that are able to multiply in an N-free culture medium are assumed as nitrogen fixing bacteria. The cultivation process was repeated to obtain pure nitrogen fixing bacteria.

### Example 3: Isolating Bacteria with the Ability of Dissolving Phosphate and Having Phosphatase Enzyme

Isolated bacteria were heated at 28° C. in a liquid medium of LB to reach the concentration of 10⁶ CFU/mL. 20 μL of liquid culture medium of LB was added to the middle of a Sperber culture medium with a diameter of 0.5 cm. pH of the sample was kept constant between 7.2 and 7.3 utilizing KOH and HCl. An exemplary Sperber culture medium may include 10 g glucose, 0.5 g yeast extract, 0.1 g CaCl₂), 0.25 g MgSO₄.7H₂O, 2.5 g Ca₃(PO₄)₂, and 15 g agar. The sample was heated at 28° C. for 2, 4, and 7 days. All the experiments were performed three times for each sample. Bacteria with the ability of dissolving phosphate may show a transparent region around their colony. 50 μg/mL of 5-Bromo-4-chloro-3-indolyl phosphate was added to Sperber medium culture to evaluate phosphatase enzyme activity. Freshly cultured bacteria were added to an LB culture medium. Samples were heated for 72 hours at 28° C. 5-Bromo-4-chloro-3-indolyl phosphate is transparent and when the phosphatase enzyme separates bromo chloro indolyl the color changes to blue. The blue color may indicate phosphate enzyme activity. Isolated bacteria with the nucleotide sequence of SEQ ID NO. 1, SEQ ID NO. 2, SEQ ID NO. 3, SEQ ID NO. 8, and SEQ ID NO. 9 show the highest ability for dissolving phosphate.

### Example 4: Isolating Bacteria with the Ability of Dissolving Potassium

Isolated bacteria were heated at 28° C. in a liquid medium of LB to reach the concentration of 10⁶ CFU/mL. 20 μL of liquid culture medium of LB was added to the middle of an Aleksandrov culture medium with a diameter of 0.5 cm. pH of the sample was kept constant between 7.0 and 7.5 utilizing NaOH and HCl. In an exemplary embodiment the Aleksandrov culture medium may contain 0.5% glucose, 0.05% magnesium sulfate heptahydrate (MgSO₄.7H₂O), 0.0005% iron (III) chloride (FeCl₃), 0.01% calcium carbonate (CaCO₃), 0.2% calcium phosphate Ca₃(PO₄)₂, 0.2% mica powder, and 15 g agar. The sample was heated at 28° C. for 2, 4, and 7 days. All the experiments were performed three times for each sample. Bacteria with the ability of dissolving potassium may show a transparent region around their colony. Potassium solution of Aleksandrov culture medium was washed utilizing HCl 0.1 M and distilled water due to high soluble potassium content of mica. 0.4 g of mica powder was dissolved in 30 mL HCl 0.1 M. the mixture was centrifuged for 5 minutes and 10000 rpm. 30 mL water was added to the precipitate and the mixture was mixed for 30 minutes. Then the mixture was centrifuged again for 5 minutes and 10000 rpm to extract the precipitate. The mica powder was dried at 80° C. Isolated bacteria with the nucleotide sequence of SEQ ID NO. 1, SEQ ID NO. 2, SEQ ID NO. 3, SEQ ID NO. 7, SEQ ID NO. 8, and SEQ ID NO. 9 may show the highest ability of dissolving potassium. Potassium solubility of 10 samples after 0, 48, and 96 hours are shown in Table 3.

TABLE 3

| | Concentration of potassium (mg/L) | | |
|---|---|---|---|
| Sample | 0 hour | 48 hours | 96 hours |
| A₁ | 2.021 | 16.4 | 43.17 |
| A₂ | 2.09 | 12.3 | 12.3 |
| A₃ | 2.08 | 16.4 | 15.38 |
| A₄ | 2.1 | 3.03 | 3.08 |
| A₅ | 2.12 | 4.96 | 4.92 |
| A₆ | 2.06 | 3.54 | 3.69 |
| A₇ | 2.05 | 6.98 | 7.15 |
| A₈ | 1.99 | 16.3 | 14.35 |
| A₉ | 1.96 | 13.32 | 15.38 |
| A₁₀ | 2.04 | 2.20 | 2.23 |
| control | 2.03 | 2.11 | 2.12 |

### Example 5: Isolating Bacteria with the Ability of Producing Plant Growth Hormones

Isolated bacteria were cultured in a liquid culture medium LB with 0.1 w/v % tryptophan to specify indole-3-acetic acid which is an auxin hormone. The mixture was heated at 28° C. for 24 hours and centrifuged at 150 rpm. After 24 hours, the mixture was centrifuged at 13000 rpm for 2 minutes. 2 mL of the supernatant was added to 2 mL of Salkowski indicator. As used herein, Salkowski indicator may contain 2 ml of 0.5M FeCl₃, and 98 ml of 35% HClO₄. The mixture was heated at 28° C. for 30 minutes in an incubator. The light absorbance of the samples was studied at 530 nm utilizing a spectrophotometer. Isolated bacteria with the nucleotide sequence of SEQ ID NO. 1, SEQ ID NO. 2, SEQ ID NO. 3, SEQ ID NO. 4, and SEQ ID NO. 8 may show the highest auxin production.

### Example 6: Quantitative Study of Dissolving Phosphate of Isolated Bacteria

100 μL of the bacteria solution with the concentration of 10⁶ CFU/mL was added to an Erlenmeyer flask containing Sperber culture medium. An exemplary Sperber culture medium may include 10 g Glucose, 0.5 g yeast extract, 0.1 g CaCl₂), 0.25 g MgSO₄.7H₂O, 2.5 g Ca₃(PO₄)₂, and 15 g agar. The mixture was heated at 28° C. and centrifuged at 100 rpm. 1000 of the mixture was collected after 0, 5, 6, 12, 24, 48, 72, and 96 hours and the samples were centrifuged at 13000 rpm for 5 minutes. 290 μL of the supernatant, 1140 μL distilled water, and 570 μL of vanadomolybdate indicator were mixed. The mixture may turn yellow which may show the ability of bacteria to dissolve phosphate. Light absorbance of the samples were detected by a spectrophotometer at 420 nm. All the analysis data regarding phosphate solubility index, potassium solubility index, production of auxin hormone, production of phosphate enzyme, production of ammonia, and growth in the N-free medium after 7 days are shown in Table 4. Phosphate and potassium solubility data of 10 samples after 2, 4, and 7 days are shown in Table 5.

TABLE 4

| | | | | Production | Production | Growth |
|---|---|---|---|---|---|---|
| | Phosphate | Potassium | Production | of | of | in the |
| | solubility | solubility | of auxin | phosphate | ammonia | N-free |
| Sample | index | index | hormone | enzyme | (μmol/mL) | medium |
| A₁ | 2.91 | 2.91 | 8.99 | + | 0.569 | + |
| A₂ | 2.45 | 3.09 | 15.18 | ++ | 0.545 | ++ |
| A₃ | 3.08 | 2.83 | 14.03 | + | 0.554 | ++ |
| A₄ | 1.12 | 1.11 | 13.58 | + | 0.589 | + |
| A₅ | 1.42 | 1.21 | 13.81 | + | 0.542 | ++ |
| A₆ | 0.95 | 1.2 | 12.23 | + | 0.501 | + |
| A₇ | 1.21 | 1.16 | 23.66 | + | 0.432 | ++ |
| A₈ | 2.15 | 2.57 | 79.19 | + | 0.569 | ++ |
| A₉ | 3.58 | 3.03 | 18.09 | + | 0.513 | + |
| A₁₀ | 1.14 | 1.1 | 11.33 | +++ | 0.285 | + |

TABLE 5

| | | Phosphate solubility capacity | | | Potassium solubility capacity | | |
|---|---|---|---|---|---|---|---|
| | Time | Halo | Colony | | Halo | Colony | |
| Sample | (day) | diameter | diameter | PSI | diameter | diameter | KSI |
| A₁ | 2 | 18 ± 3 | 8 ± 0 | 2.25 ± 0.37 | 17 ± 2.6 | 8 ± 0 | 2.12 ± 0.33 |
| | 4 | 20 ± 2 | 8 ± 0 | 2.5 ± 0.25 | 19 ± 3.6 | 8 ± 0 | 2.37 ± 0.45 |
| | 7 | 23.3 ± 0.5 | 8 ± 0 | 2.91 ± 0.07 | 23.3 ± 1.5 | 8 ± 0 | 2.91 ± 0.19 |
| A₂ | 2 | 22 ± 2 | 10 ± 0 | 2.2 ± 0.2 | 24 ± 1 | 10 ± 0 | 2.4 ± 0.1 |
| | 4 | 23.33 ± 1.52 | 10 ± 0 | 2.33 ± 0.15 | 27.33 ± 1.15 | 10 ± 0 | 2.73 ± 0.11 |
| | 7 | 27.33 ± 3.05 | 11 ± 0 | 2.48 ± 0.27 | 34 ± 1 | 11 ± 0 | 3.09 ± 0.09 |
| A₃ | 2 | 18.33 ± 2.08 | 8 ± 0 | 2.29 ± 0.26 | 18.33 ± 2.88 | 8 ± 0 | 2.29 ± 0.36 |
| | 4 | 20 ± 2 | 8 ± 0 | 2.5 ± 0.25 | 21.66 ± 4.16 | 8 ± 0 | 2.7 ± 0.52 |
| | 7 | 24.66 ± 0.57 | 8 ± 0 | 3.8 ± 0.07 | 22.66 ± 3.21 | 8 ± 0 | 2.83 ± 0.4 |
| A₄ | 2 | 8.5 ± 0.1 | 8 ± 0 | 1.06 ± 0.01 | 8.46 ± 0.05 | 8 ± 0 | 1.05 ± 0.007 |
| | 4 | 9.2 ± 0.26 | 8 ± 0 | 1.15 ± 0.03 | 9.06 ± 0.11 | 8 ± 0 | 1.13 ± 0.01 |
| | 7 | 10.16 ± 0.28 | 9 ± 0 | 1.12 ± 0.03 | 10.06 ± 0.11 | 9 ± 0 | 1.11 ± 0.01 |
| A₅ | 2 | 9 ± 0 | 8 ± 0 | 1.125 ± 0 | 9.33 ± 0.28 | 8 ± 0 | 1.16 ± .03 |
| | 4 | 10.33 ± 0.57 | 8 ± 0 | 1.29 ± 0.07 | 9.33 ± 0.76 | 8 ± 0 | 1.22 ± 0.09 |
| | 7 | 11.83 ± 0.28 | 8.3 ± 0.57 | 1.42 ± 0.08 | 10.5 ± 0.5 | 8.6 ± 0.57 | 1.21 ± 0.04 |
| A₆ | 2 | 8.26 ± 0.2 | 8 ± 0 | 1.03 ± 0.02 | 9 ± 0 | 8 ± 0 | 1.25 ± 0 |
| | 4 | 9.03 ± 0.05 | 8 ± 0 | 1.12 ± 0.007 | 9.56 ± 0.4 | 8.66 ± 0.57 | 1.1 ± 0.04 |
| | 7 | 9.16 ± 0.28 | 6.9 ± 0.57 | 1.21 ± 0.006 | 11.16 ± 0.28 | 9.33 ± 0.57 | 1.2 ± 0.09 |
| A₇ | 2 | 10 ± 0 | 9 ± 0 | 1.11 ± 0 | 9.33 ± 0.28 | 8.66 ± 0.57 | 1.07 ± 0.04 |
| | 4 | 10.33 ± 0.28 | 6.9 ± 0.28 | 1.12 ± 0.06 | 10.16 ± 0.28 | 8.66 ± 0.57 | 1.71 ± 0.06 |
| | 7 | 11.16 ± 0.28 | 6.9 ± 0.28 | 0.95 ± 0.05 | 10.5 ± 0 | 9 ± 0 | 1.16 ± 0 |
| A₈ | 2 | 24.33 ± 0.57 | 12.33 ± 0.05 | 2.06 ± 0.54 | 23.66 ± 0.57 | 9.33 ± 0.57 | 2.53 ± 0.1 |
| | 4 | 27.33 ± 2.51 | 13.663 ± 1.25 | 2.008 ± 0.18 | 26.66 ± 0.57 | 11.33 ± 0.57 | 2.57 ± 0.24 |
| | 7 | 29.33 ± 2.51 | 13.66 ± 1.25 | 2.15 ± 0.19 | 30 ± 1.73 | 11.66 ± 0.57 | 2.51 ± 0.36 |
| A₉ | 2 | 21 ± 3.46 | 7.66 ± 0.57 | 2.76 ± 0.58 | 18.33 ± 1.52 | 7.33 ± 0.57 | 2.57 ± 0.55 |
| | 4 | 23.33 ± 3.05 | 7.66 ± 0.57 | 3.05 ± 0.49 | 21 ± 3.6 | 7.66 ± 0.57 | 3.03 ± 0.37 |
| | 7 | 27.33 ± 1.15 | 7.66 ± 0.57 | 3.58 ± 0.38 | 23.33 ± 2.51 | 7.66 ± 0.57 | 1.04 ± 0.03 |
| A₁₀ | 2 | 8.66 ± 2.28 | 8.16 ± 028 | 1.06 ± 0.002 | 8.5 ± 0 | 8.16 ± 28 | 1.04 ± 0.03 |
| | 4 | 9 ± 0 | 8.16 ± 0.28 | 1.1 ± 0.03 | 8.9 ± 0.1 | 8.16 ± 28 | 1.09 ± 0.02 |
| | 7 | 9.33 ± 0.28 | 8.16 ± 0.28 | 1.14 ± 0.003 | 9 ± 0 | 8.16 ± 28 | 1.1 ± 0.03 |

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for description and not of limitation, such that the terminology or phraseology of the present specification is to be interpreted by the skilled artisan in light of the teachings and guidance.

The breadth and scope of the present disclosure should not be limited by any of the above-described exemplary embodiments but should be defined only in accordance with the following claims and their equivalents.

Throughout this specification and the claims which follow, unless the context requires otherwise, the word “comprise”, and variations such as “comprises” or “comprising”, will be understood to imply the inclusion of a stated integer or step or group of integers or steps but not to the exclusion of any other integer or step or group of integers or steps. Moreover, the word “substantially” when used with an adjective or adverb is intended to enhance the scope of the particular characteristic; e.g., substantially planar is intended to mean planar, nearly planar and/or exhibiting characteristics associated with a planar element. Further use of relative terms such as “vertical”, “horizontal”, “up”, “down”, and “side-to-side” are used in a relative sense to the normal orientation of the apparatus.

What is claimed is:

1. A biofertilizer, comprising:
a first component comprising an isolated bacterium blended with a first carrier, the isolated bacterium comprising a nucleic acid sequence identical to at least one of SEQ ID NO. 1 and SEQ ID NO. 3; and
a second component comprising a pair of isolated bacteria blended with a second carrier, the pair of isolated bacteria comprising nucleic acid sequences identical to nucleic acid sequences selected from the group consisting of SEQ ID NO. 1, SEQ ID NO. 3, and SEQ ID NO. 5.

2. A biofertilizer, comprising:
a first component comprising an isolated bacterium blended with a first carrier, the isolated bacterium comprising a nucleic acid sequence identical to at least one of SEQ ID NO. 1, SEQ ID NO. 2, SEQ ID NO. 3, SEQ ID NO. 8, and SEQ ID NO. 9; and
a second component comprising a pair of isolated bacteria blended with a second carrier, the pair of isolated bacteria comprising nucleic acid sequences identical to nucleic acid sequences selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, and SEQ ID NO. 10.

3. The biofertilizer of claim 2, wherein the first carrier comprises:
2-3 wt. % of a corn steep liquor, based on the total weight of the first component;
0.5-1 wt. % of sugar beet molasses, based on the total weight of the first component;
1-10 wt. % of glycerin, based on the total weight of the first component; and
water.

4. The biofertilizer of claim 3, wherein the first component comprises 0.5 wt. % to 10 wt. % of the isolated bacterium based on the total weight of the first component.

5. The biofertilizer of claim 4, wherein the second carrier comprises:
2-3 wt. % of a corn steep liquor, based on the total weight of the second component;
0.5-1 wt. % of sugar beet molasses, based on the total weight of the second component;
1-10 wt. % of glycerin, based on the total weight of the second component; and
water.

6. The biofertilizer of claim 5, wherein the second component comprises 0.5 wt. % to 10 wt. % of the pair of isolated bacteria based on the total weight of the second component.

7. The biofertilizer of claim 2, wherein the first carrier comprises:
0.5-1.5 wt. % of pearlite, based on the total weight of the first component;
0.5-1.5 wt. % of bentonite, based on the total weight of the first component;
a filler comprising one of:
a first filler comprising 0.5-1 wt. % of soybean meal and 50 wt. % of bagasse, based on the total weight of the first component, and
a second filler comprising 50 wt. % of soybean meal and 0.5-1 wt. % of bagasse, based on the total weight of the first component; and
water.

8. The biofertilizer of claim 7, wherein the first component comprises 1 wt. % to 10 wt. % of the isolated bacterium based on the total weight of the first component.

9. The biofertilizer of claim 8, wherein the second carrier comprises
0.5-1.5 wt. % of pearlite, based on the total weight of the second component;
0.5-1.5 wt. % of bentonite, based on the total weight of the second component;
a filler comprising one of:
a first filler comprising 0.5-1 wt. % of soybean meal and 50 wt. % of bagasse, based on the total weight of the second component, and
a second filler comprising 50 wt. % of soybean meal and 0.5-1 wt. % of bagasse, based on the total weight of the second component; and
water.

10. The biofertilizer of claim 9, wherein the second component comprises 1 wt. % to 10 wt. % of the pair of isolated bacteria based on the total weight of the second component.

11. The biofertilizer of claim 2, wherein the isolated bacterium comprises at least one of Enterobacteriaceae bacterium, pantoea sp. strain SPC23, kluyvera sp. strain PS20, Enterobacter ludwigii strain DM27, and pectobacterium cypripedii strain ICMP 1591.

12. The biofertilizer of claim 3, wherein the pair of isolated bacteria are selected from a group consisting of Enterobacteriaceae bacterium, pantoea sp. strain SPC23, kluyvera sp. strain PS20, Enterobacter ludwigi strain SNU502, Lelliottia amnigena strain NCTC12124, klebsiella sp. strain MWX2, Enterobacter ludwigii strain DM27, pectobacterium cypripedii strain ICMP 1591, and stenotrophomonas sp. G4.

13. A method for producing a biofertilizer, the method comprising:
forming a first component by:
forming a first mixture by blending an isolated bacterium with a first carrier, the isolated bacterium comprising a nucleic acid sequence identical to at least one of SEQ ID NO. 1, SEQ ID NO. 2, SEQ ID NO. 3, SEQ ID NO. 8, and SEQ ID NO. 9; and
heating the first mixture at a temperature between 28° C. and 30° C.; and
forming a second component by:
forming a second mixture by blending a pair of isolated bacteria with a second carrier, the pair of isolated bacteria comprising nucleic acid sequences identical to nucleic acid sequences selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, and SEQ ID NO. 10;
heating the second mixture at a temperature between 28° C. and 30° C.; and
mixing the first component and the second component.

14. The method of claim 13, wherein forming the first mixture comprises blending the isolated bacterium with the first carrier with a concentration of the isolated bacterium between 1 wt. % and 10 wt. % of the total weight of the first component, the first carrier comprising:
2-3 wt. % of a corn steep liquor, based on the total weight of the first component;
0.5-1 wt. % of sugar beet molasses, based on the total weight of the first component;
1-10 wt. % of glycerin, based on the total weight of the first component; and
water.

15. The method of claim 14, wherein forming the second mixture comprises blending the pair of isolated bacteria with the second carrier with a concentration of the pair of isolated bacteria between 1 wt. % and 10 wt. % of the total weight of the second component, the second carrier comprising:
2-3 wt. % of a corn steep liquor, based on the total weight of the second component;
0.5-1 wt. % of sugar beet molasses, based on the total weight of the second component;
1-10 wt. % of glycerin, based on the total weight of the second component; and
water.

16. The method claim 15, further comprising adjusting pH of the first component and the second component at a pH between 7 and 9.

17. The method of claim 13, wherein forming the first mixture comprises blending the isolated bacterium with the first carrier with a concentration of the isolated bacterium between 0.5 wt. % and 10 wt. % of the total weight of the first component, the first carrier comprising:
0.5-1.5 wt. % of pearlite, based on the total weight of the first component;
0.5-1.5 wt. % of bentonite, based on the total weight of the first component;
a filler comprising one of:
a first filler comprising 0.5-1 wt. % of soybean meal and 50 wt. % of bagasse, based on the total weight of the first component, and
a second filler comprising 50 wt. % of soybean meal and 0.5-1 wt. % of bagasse, based on the total weight of the first component; and
water.

18. The method of claim 17, wherein forming the second mixture comprises blending the pair of isolated bacteria with the second carrier with a concentration of the pair of isolated bacteria between 0.5 wt. % and 10 wt. % of the total weight of the second component, the second carrier comprising:
0.5-1.5 wt. % of pearlite, based on the total weight of the second component;
0.5-1.5 wt. % of bentonite, based on the total weight of the second component;
a filler comprising one of:
a first filler comprising 0.5-1 wt. % of soybean meal and 50 wt. % of bagasse, based on the total weight of the second component, and
a second filler comprising 50 wt. % of soybean meal and 0.5-1 wt. % of bagasse, based on the total weight of the second component; and
water.

19. The method claim 18, further comprising adjusting pH of the first component and the second component at a pH between 7 and 9.

20. The method of claim 13, further comprising mixing the first component and the second component with a weight ratio of the first component to the second component between 0.5 and 2 (first component/second component).
